(19) United States
(12) Patent Application Publication
(10) Pub. No.: US 2008/0233643 A1
(43) Pub. Date: Sep. 25, 2008

(54) Differentiation of Cd34 Positive Cell to Megakaryocyte and Multiplication

(75) Inventors: Takuya Matsunaga, Hokkaido (JP); Hirofumi Hamada, Hokkaido (JP); Yoshio Niitsu, Hokkaido (JP)

Correspondence Address: FOLEY AND LARDNER LLP;SUITE 500, 3000 K STREET NW, WASHINGTON, DC, 20007, US

(73) Assignee: Renomedix Institute Inc.

(21) Appl. No.: 10/585,763

(22) PCT Filed: Mar. 17, 2004
(86) PCT No.: PCT/JP04/03552

§ 371 Date: Jul. 12, 2006

(30) Foreign Application Priority Data
Jan. 14, 2004 (JP) 2004-007169

(51) Int. Cl. C12N 5/06

(52) U.S. Cl. 435/373; 435/325

(57) ABSTRACT

An object of the present invention is to provide a method for more efficiently differentiating CD34⁺ cells derived from umbilical cord blood into megakaryocytic lineage cells and for generating platelets. Coculture of CD34⁺ cells derived from umbilical cord blood with immortalized stromal cells in the presence of cytokines has enabled cell proliferation up to the 100,000-fold level, which has been impossible to achieve to date.

### TECHNICAL FIELD

The invention of the present application relates to the proliferation and/or differentiation of CD34⁺ cells derived from umbilical cord blood into megakaryocytes. More specifically, the invention of the present application relates to the proliferation and/or differentiation of CD34⁺ cells derived from umbilical cord blood into megakaryocytes using immortalized stromal cells.

### BACKGROUND ART

Hematopoietic stem cell transplantation is performed for intractable blood diseases such as leukemia. The stem cell transplantation requires a large quantity of blood stem cells. Hematopoietic stem cells can be recovered from the bone marrow, peripheral blood, or umbilical cord blood through bone marrow aspiration, recovery of peripheral blood stem cells, or the like. Of these, stem cells derived from umbilical cord blood are characterized by having proliferative ability higher than that of stem cells derived from the bone marrow and having low immunoreactivity that leads to low possibility of rejection. Accordingly, stem cell transplantation using umbilical cord blood is thought to be an effective method, when no appropriate donors can be found (Non-patent document 1: New England Journal of Medicine Vol. 335, pp. 157-166).

Meanwhile, the number of stem cells that can be recovered at a time from umbilical cord blood is limited. Hence, cell proliferation methods have been studied. It has been reported that the number of human stem cells derived from the bone marrow could be doubled through stimulation with SCF (stem cell factor), FL (Flt-3/Flk-2 ligand), G-CSF (Granulocyte Colony Stimulating Factor), IL-3, and IL-6 (Non-patent document 2: Proc. Natl. Acad. Sci. U.S.A. Vol. 94, 13648). Regarding stem cells derived from umbilical cord blood, it has also been reported that such cells were proliferated ex vivo using Flt-3, FL, SCF, TPO (thrombopoietin), and IL-3 (interleukin 3, hereinafter also referred to as IL3) (Non-patent document 3: Stem Cell 2001 Vol. 19, pp. 313-320).

Furthermore, it is known that umbilical cord blood stem cell transplantation has a drawback compared with bone marrow transplantation or peripheral blood stem cell transplantation in that it takes a great deal of time to increase the numbers of erythrocytes, leukocytes, and platelets, and particularly the number of platelets (Non-patent document 4: The New England Journal of Medicine Vol. 339, pp. 1565-1577).

Non-patent document 1: New England Journal of Medicine Vol. 335, pp. 157-166

Non-patent document 2: Proc. Natl. Acad. Sci. U.S.A. Vol. 94, pp. 13648-13653

Non-patent document 3: Stem Cell 2001 Vol. 19, pp. 313-320

Non-patent document 4: The New England Journal of Medicine Vol. 339, pp. 1565-1577

### DISCLOSURE OF THE INVENTION

In ex vivo proliferation of stem cells derived from umbilical cord blood, the proliferation and differentiation of such cells into megakaryocytic lineage cells have also been attempted. For example, in the case of proliferation (amplification) with the use of TPO and SCF, it has been reported that megakaryocytic lineage cells had increased 210-fold by day 14 after culture and 240-fold by day 21 after culture. To achieve further amplification, 4 weeks of amplification of umbilical cord blood cells was attempted, followed by the amplification and differentiation of the resultant cells into megakaryocytic lineage cells. However, the cells could be amplified approximately 40-fold by day 14 even with the use of IL-3-SCF-IL-6-TPO. This was still insufficient in terms of maximum amplification rate. Moreover, it has also been reported that when amplified cord blood cells by culture are differentiated to megakaryocytic lineage cells, the megakaryocytic lineage cells are so immature as to be able to form tetraploids at best. It has also been reported that even when these megakaryocytic lineage cells were transplanted into NOD/SCID mice, human CD41⁺ cells were observed only temporarily (Hematologica Vol. 88, pp. 379-387 (April 2003)).

Hence, an object of the invention of the present application is to provide a method for more efficiently differentiating megakaryocytic lineage cells and for generating platelets from stem cells derived from umbilical cord blood.

The inventors of the present application have achieved cell proliferation up to the 100,000-fold level (which has been impossible to achieve to date) through coculture of stem cells derived from umbilical cord blood with immortalized stromal cells in the presence of cytokines.

This description includes part or all of the contents as disclosed in the description and/or drawings of Japanese Patent Application No. 2004-007169, which is priority document of the present application.

### BRIEF DESCRIPTION OF THE DRAWINGS

FIG. 1 shows the outline of the example of the method.

FIG. 2 shows the total cell counts obtained in the example.

FIG. 3 shows the percentages of CD41⁺ cells obtained in the example.

FIG. 4 shows the proliferation degrees of CD41⁺ cells obtained in the example.

FIG. 5 shows images from days 14, 21, 25, and 28 after culture as obtained by May-Giemsa staining and a phase contrast microscopic image from day 28 (lower right) after culture in the example.

### BEST MODE FOR CARRYING OUT THE INVENTION

The invention of the present application encompasses a method for efficiently preparing CD41⁺ cells (megakaryocytic lineage cells), which comprises coculturing immortalized stromal cells with CD34⁺ cells.

CD34⁺ cells that are used in the invention of the present application can be obtained from bone marrow, peripheral blood, and umbilical cord blood. Furthermore, CD34⁺ cells that have been differentiated from ES cells and CD34⁺ cells that have been differentiated from other (stem) cells can also be used. Preferably, CD34⁺ cells derived from umbilical cord blood can be used.

After separation of mononuclear cells from umbilical cord blood cells, CD34⁺ cells can be separated from the mononuclear cells by methods using a CD34-specific antibody, superparamagnetic microbeads, miniMACS, or multi-MACS.

The method of the present application encompasses: (1) a method for coculturing CD34⁺ cells with immortalized stromal cells; (2) the step of coculturing CD34⁺ cells with immortalized stromal cells and a method for further culturing CD34⁺ cells in the presence of a cytokine(s) after removal of the immortalized stromal cells; and (3) a method for culturing CD34⁺ cells and immortalized stromal cells in the presence of a cytokine(s).

Preferable immortalized stromal cells that are used in the invention of the present application are mammalian stromal cells, particularly immortalized stromal cells prepared by immortalization of human-derived stromal cells. Preferably, human bone marrow stromal cells (hereinafter referred to as hTERT stromal cells) having the hTERT gene (disclosed in WO03/038076) introduced therein can be used. In addition, the hTERT sequence is disclosed in Science 277, pp. 955-959, for example.

hTERT stromal cells can be prepared as follows, for example.

An hTERT EcoRV-SalI fragment obtained by PCR from pCI-Neo-hTERT-HA is cloned into pBABE-hygro, thereby constructing pBABE-hygro-hTERT (Proc. Natl. Acad. Sci. U.S.A. Vol. 95, pp. 14723-14728).

Next, ψCRIP packaging cells (Proc. Natl. Acad. Sci. U.S.A., 90: 3539-3543, 1993) are prepared using BOSC23 packaging cells (Proc. Natl. Acad. Sci. U.S.A., 90: 8392-8396, 1993). The above vector is proliferated by retrovirus-producing cells (ψCRIP/P131).

On the day before infection, stromal cells are seeded again, a medium for retrovirus-producing ψCRIP/P131 is exchanged with fresh medium, and then the cells are cultured. Stromal cells are then infected with recombinant retrovirus vectors produced in the supernatant. 4 hours later, the cells are further cultured for 2 days after exchanging the culture supernatant with fresh medium. Subsequently, pBABE-hygro-hTERT is subjected to 5 days of drug selection using 100 μg/ml hygromycin, so that hTERT stromal cells can be prepared.

When the invention of the present application is prepared in 2 stages (a 1^{st} step of coculturing CD34⁺ cells with immortalized stromal cells and a 2^{nd} step of culturing the resultant cells in the presence of a cytokine(s) after removal of the immortalized stromal cells), preferably, the 1^{st} step for coculturing CD34⁺ cells with immortalized stromal cells can be continued until the CD34⁺ cells are differentiated into megakaryocytic lineage progenitor cells (hematopoietic progenitor cells that can be differentiated into megakaryocytic lineage cells). Specifically, the 1^{st} step can be continued for 5 to 21 days or 10 to 20 days (e.g., for 14 days). Furthermore, as cytokines that are caused to be present in the 1^{st} step, SCF, TPO, and a Flt-3/Flk-2 ligand can be used in combination.

The 2^{nd} step for culturing the resultant cells can be continued until megakaryocytic lineage progenitor cells are differentiated into megakaryocytic lineage cells (CD41⁺ cells). Specifically, the 2^{nd} step can be continued for 7 to 21 days or 12 to 16 days (e.g., for 14 days). Examples of cytokines that are caused to be present in the 2^{nd} step include: (1) TPO; (2) TPO and interleukin (IL) 3; (3) TPO, IL1, IL3, IL6, and IL11, and (4) TPO, IL1, IL3, IL6, IL11, SCF, and FL.

### EXAMPLE 1

### Examination Concerning Proliferation and/or Differentiation of CD34⁺ Cells Derived from Umbilical Cord Blood into Megakaryocytes Using Immortalized Stromal Cells

### (1) Outline:

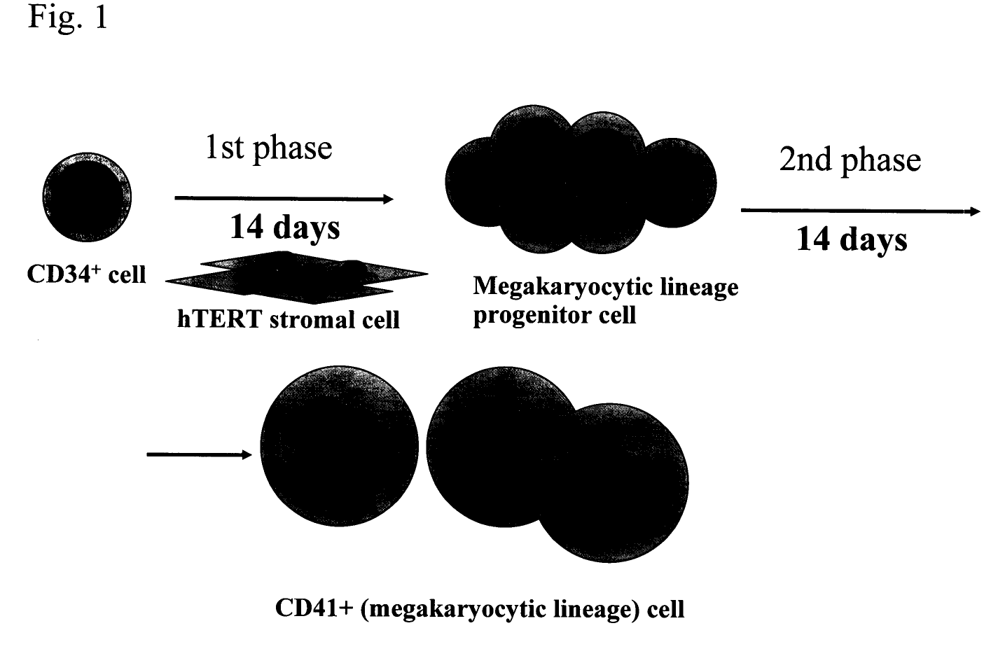

CD34⁺ cells derived from umbilical cord blood were proliferated through 14 days of coculture with immortalized stromal cells (hTERT stromal cells). The thus proliferated CD34⁺ cells were further subjected to another 14 days of liquid culture in the presence of cytokines including thrombopoietin (TPO). Therefore, the cells were efficiently proliferated and/or differentiated into CD41⁺ (megakaryocytic lineage) cells (see FIG. 1).

### (2) Materials and Methods:

CD34⁺ cells used herein were separated from umbilical cord blood using a MACS separation kit (Miltenyi Biotec).

Immortalized stromal cells (hTERT stromal cells) used herein were human bone marrow stromal cells wherein the hTERT gene had been introduced disclosed in WO03/038076.

Proliferation and differentiation of CD41⁺ (megakaryocytic lineage) cells were performed in 2 stages.

(a) 1^{st} step (on days 0 to 14 after separation of CD34⁺ cells): 75 cm² flasks were used. The CD34⁺ cells separated as described above and immortalized stromal cells (hTERT stromal cells) that had reached confluency were both suspended at a concentration of 5×10²/9 ml in a medium X-VIVO 10 supplemented with 10 ng/ml stem cell factor (SCF), 50 ng/ml thrombopoietin (TPO), and 50 ng/ml Flt-3/Flk-2 ligand (FL). The cells were then cultured for 14 days.

On day 7 after culture, 9 ml of a medium having the same composition as the above medium was added.

(b) 2^{nd} step (days 14 to 28 after the start of culture): 6-well plates were used. Megakaryocytic lineage progenitor cells comprising the suspended cells that had been prepared in the 1^{st} step were suspended at a concentration of 2×10⁵/ml in 4 culture systems of media X-VIVO 10 supplemented with: (1) TPO; (2) TPO and interleukin (IL) 3; (3) TPO, IL1, IL3, IL6, and IL11; and (4) TPO, IL1, IL3, IL6, IL11, SCF, and FL, respectively. The cells were subjected to 14 days of liquid culture. Cytokine concentrations were 50 ng/ml TPO, 25 ng/ml IL1, 25 ng/ml IL3, 25 ng/ml IL6, 25 ng/ml IL11, 25 ng/ml SCF, and 50 ng/ml FL, respectively.

The cells were recovered on day 21. The media were exchanged with fresh media. All the cells were cultured under conditions of 37° C. and 5% CO₂. The cells were recovered on days 21 and 28, and then the total cell counts were determined. Cell surface character was analyzed using flow cytometory. Furthermore, cytospin samples were prepared and then subjected to May-Giemsa staining to observe cell morphology.

### (3) Results:

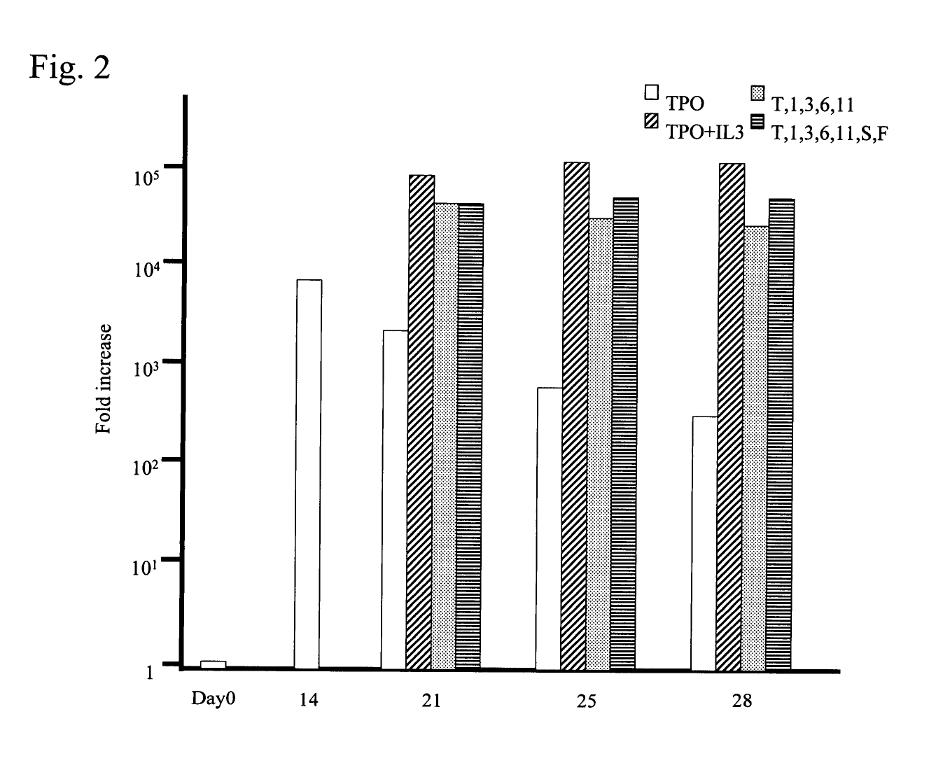

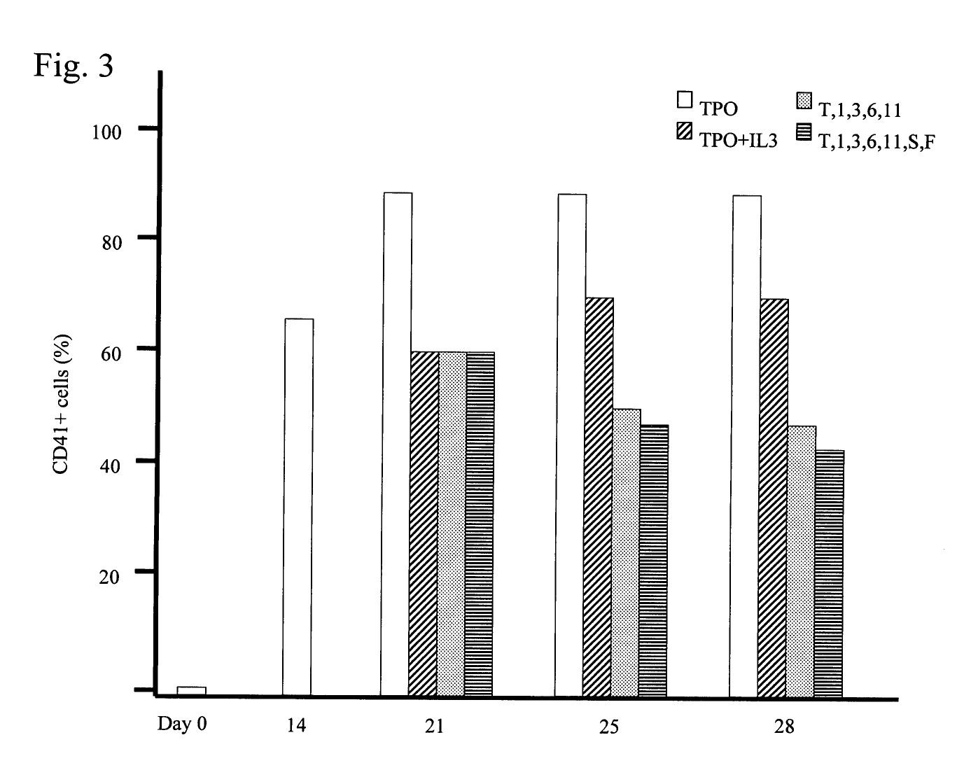

FIG. 2 shows the total cell counts obtained by culture.
(a) As a result of coculture in the 1^{st} step, the total cell count was amplified 8,000-fold from 5×10² to 4×10⁶.
(b) Furthermore, as a result of liquid culture in the 2^{nd} step: (1) in the case of adding TPO, the cells were amplified to a total cell count of 1.7×10⁵ (330-fold amplification); (2) in the case of adding TPO and IL3, the cells were amplified to a total cell count of 5.2×10⁷ (104,000-fold amplification); (3) in the case of adding TPO, IL1, IL3, IL6, and IL11, the cells were amplified to a total cell count of 1.5×10⁷ (30,000-fold amplification); and (4) in the case of adding TPO, IL1, IL3, IL6, IL11, SCF, and FL, the cells were amplified to a total cell count of 2.8×10⁷ (56,000-fold amplification).
(c) FIG. 3 shows the results of analysis using flow cytometry. On day 14, the percentage of CD41⁺ cells was 66%. On day 21, the percentages of CD41⁺ cells were: (1) 89% in the case of TPO; (2) 60% in the case of TPO and IL3; (3) 60% in the case of TPO, IL1, IL3, IL6, and IL11; and (4) 60% in the case of TPO, IL1, IL3, IL6, IL11, SCF, and FL.

On day 25, the percentages of CD41⁺ cells were: (1) 81% in the case of TPO; (2) 70% in the case of TPO and IL3; (3) 50% in the case of TPO, IL1, IL3, IL6, and IL11; and (4) 45% in the case of TPO, IL1, IL3, IL6, IL11, SCF, and FL.

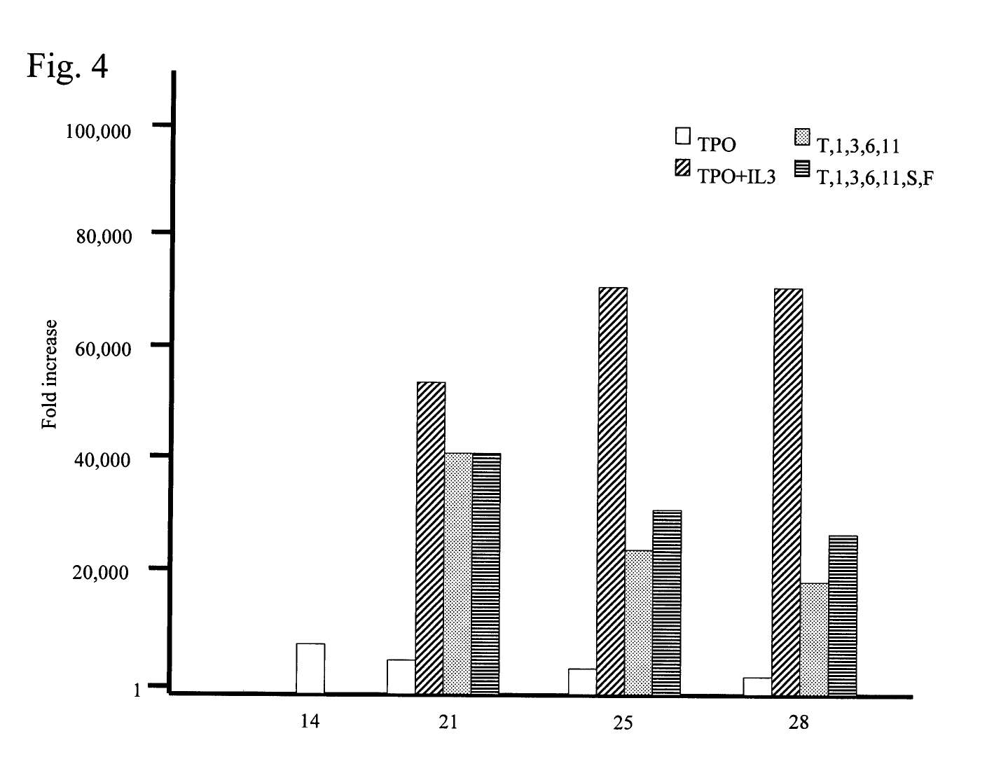

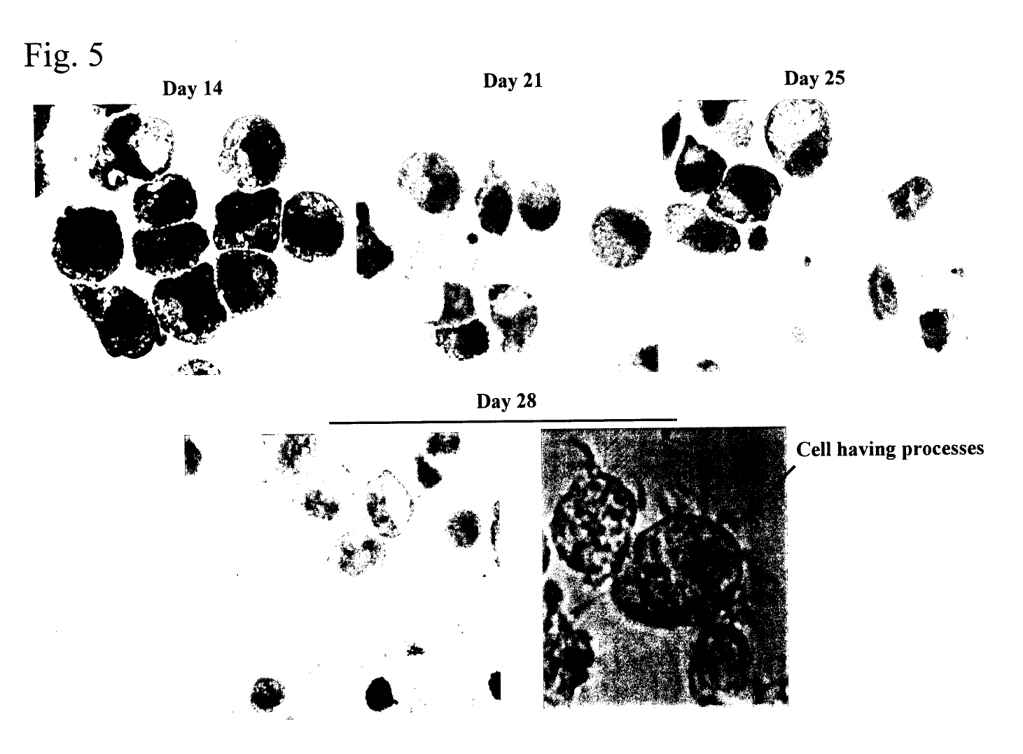

Furthermore, on day 28, the percentages of CD41⁺ cells were: (1) 81% in the case of TPO; (2) 70% in the case of TPO and IL3; (3) 44% in the case of TPO, IL1, IL3, IL6, and IL11; and (4) 39% in the case of TPO, IL1, IL3, IL6, IL11, SCF, and FL.
(d) Specifically, as shown in FIG. 4, 5×10² CD34⁺ cells could be proliferated and then differentiated into CD41⁺ cells, the cell counts of which were amplified by day 28 after the start of culture as follows: (1) 260-fold amplification in the case of TPO; (2) 73,000-fold amplification in the case of TPO and IL3; (3) 15,000-fold amplification in the case of TPO, IL1, IL3, IL6, and IL11; and (4) 24,000-fold amplification in the case of TPO, IL1, IL3, IL6, IL11, SCF, and FL.
(e) FIG. 5 shows images as obtained by performing May-Giemsa staining and a phase contrast microscopic image.

On day 14 after the start of culture, most cells were thought to be juvenile progenitor cells.

On day 21 after the start of culture, the percentages of slightly large cells were: (1) 35% in the case of using TPO; (2) 25% in the case of using TPO and IL3; (3) 20% in the case of using TPO, IL1, IL3, IL6, and IL11; and (4) 10% in the case of using TPO, IL1, IL3, IL6, IL11, SCF, and FL.

On day 25 after the start of culture, the percentages of large cells were: (1) 60% in the case of using TPO; (2) 50% in the case of using TPO and IL3; (3) 40% in the case of using TPO, IL1, IL3, IL6, and IL11; and (4) 30% in the case of using TPO, IL1, IL3, IL6, IL11, SCF, and FL.

On day 28 after the start of culture, the percentages of large cells were: (1) 70% in the case of using TPO; (2) 65% in the case of using TPO and IL3; (3) 45% in the case of using TPO, IL1, IL3, IL6, and IL11; and (4) 30% in the case of using TPO, IL1, IL3, IL6, IL11, SCF, and FL.

Increases in large cells were observed with time in each culture system.

When large cells were observed on day 28 after the start of culture with the use of the phase contrast microscopic image, the cells were found to have processes. Thus, these cells were thought to be proplatelets.
(f) As described above, technology was established by which megakaryocytic lineage cells can be proliferated in vitro in a large quantity using immortalized stromal cells.

All publications, patents, and patent applications cited herein are incorporated herein by reference in their entirety.

### INDUSTRIAL APPLICABILITY

According to the method of the present invention, the proliferation and/or differentiation of CD34⁺ cells derived from umbilical cord blood into megakaryocytic lineage cells enables treatment of thrombocytopenia or the like induced by chemotherapy, for example.

1. A method for preparing a megakaryocytic lineage cell, which comprises coculturing a CD34⁺ cell with an immortalized stromal cell.

2. The method according to claim 1, wherein a CD34⁺ cell and an immortalized stromal cell are cocultured in the presence of a cytokine.

3. The method according to claim 1, which comprises: the step (1^{st} step) of coculturing a CD34⁺ cell with an immortalized stromal cell in the presence of a cytokine; and the step (2^{nd} step) of culturing the resultant cell after removal of the immortalized stromal cell in the presence of a cytokine.

4. The method according to claim 1, which comprises: the step (1^{st} step) of cocultuirng a CD34⁺ cell with an immortalized stromal cell in the presence of a cytokine, so as to prepare a megakaryocytic lineage progenitor cell that is a hematopoietic progenitor cell capable of differentiating into a megakaryocytic lineage cell; and the step (2^{nd} step) of culturing the megakaryocytic lineage progenitor cell in the presence of a cytokine after removal of the immortalized stromal cell.

5. The method according to claim 3 or 4, wherein the cytokine used in the 1^{st} step is an SCF, TPO, or Flt-3/Flk-2 ligand.

6. The method according to claim 3 or 4, the cytokine(s) used in the 2^{nd} step is/are (1) TPO, (2) TPO and IL3, (3) TPO, IL1, IL3, IL6 and IL11, or (4) TPO, IL1, IL3, IL6, IL11, SCF, and FL.

7. The method according to any one of claims 1 to 6, wherein a CD34⁺ cell separated from umbilical cord blood is used.

8. A megakaryocytic lineage cell, which is prepared by the method according to any one of claims 1 to 7.
